(19) United States
(12) Patent Application Publication
(10) Pub. No.: US 2007/0026386 A1
(43) Pub. Date: Feb. 1, 2007

(54) Method for the detection of newly acquired hiv infection

(76) Inventors: Kim Margaret Wilson, Newport (AU); Elizabeth Irene Mary Johnson, North Fitzroy (AU)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/535,787

(22) PCT Filed: Nov. 24, 2003
(86) PCT No.: PCT/AU03/01569

§ 371 Date: May 19, 2006

(30) Foreign Application Priority Data
Nov. 22, 2002 (AU) 2002952817

(51) Int. Cl. C12Q 1/70

(52) U.S. Cl. 435/5; 435/6

(57) ABSTRACT

The present invention relates to the detection of a human immunodeficiency virus (HIV) infection. More particularly, the present invention relates to methods of distinguishing between early antibody responsesto HIV infection from matured responses. In one aspect, the present invention provides a method of distinguishing between a recent and an established HIV infection in a subject, the method comprising determining the immunoreactivity of IgG₃ antibodies of the subject to an HIV antigen, wherein elevated IgG₃ immunoreactivity indicates that the subject has recently been infected with HIV.

### FIELD OF THE INVENTION

The present invention relates to the detection of a human immunodeficiency virus (HIV) infection. More particularly, the present invention relates to methods of distinguishing between early antibody responses to HIV infection from matured responses. Accordingly, the present invention provides a means of defining early infection.

### BACKGROUND OF THE INVENTION

An assay that is capable of distinguishing between recently acquired and established HIV infection is essential to establish incidence for epidemiological surveys and also for monitoring new infections in upcoming vaccine trials. Currently this distinction can only be made by using modified assays that are thought to differentiate between low and high affinity HIV specific antibodies. The use of these assays is based on the premise that the immune response to an infection “matures” following prolonged exposure to virus, resulting in the production of antibodies which increase in concentration and progressively increase in affinity to the major antigens.

One of these modified assays is a “detuned” version of the first generation assays that uses HIV viral lysate as the antigen in an enzyme immunoassay (EIA) format. By decreasing incubation times and increasing the sample dilution it has been assumed that only samples containing high affinity antibodies will remain reactive in the “detuned” version of these assays (Janssen et al. 1998; Gouws et al. 2002). Thus when conducted in conjunction with an EIA that recognises low affinity antibody, the relative maturity of the antibody response is assessed. An alternative type of assay involves the use of chaotropic agents (e.g. 8M urea) to selectively dissociate specific antibodies with low affinity binding. The assay is performed in an EIA format and the denaturant is applied to the plate following addition of the plasma sample. It is assumed that only samples containing high affinity antibodies will retain the ability to bind following this treatment (Chargelegue et al. 1993).

The basic principles behind these assays have not been thoroughly investigated and many laboratories have encountered contradictory results depending on which method and which antigens they have used (Chargelegue et al. 1995; Binley et al. 1997). A number of variables including the composition of the chosen antigen, the antibody titre in each individual patient's sample and the antibody subclass distribution within the sample may contribute to the overall results reported for these types of assays.

There is a need for assays that can identify individuals who have been recently infected with the human immunodeficiency virus. Such assays are useful for epidemiological purposes, for monitoring the success of vaccine trials, and for determining the stage of infection for treatment purposes.

### SUMMARY OF THE INVENTION

The present inventors analysed the antibody responses involved in the maturation of the humoral response to HIV infection in panels of sera taken during and beyond seroconversion. By determining the individual specificity of antibodies to HIV polypeptides the inventors surprisingly observed a transient peak of IgG₃ immunoreactivity which occurs only in early infection or before the response matures. Thus, the inventors found that the IgG₃ response can be used to distinguish recent from established HIV infections.

Accordingly, in a first aspect the present invention provides a method of distinguishing between a recent and an established HIV infection in a subject, the method comprising determining the immunoreactivity of IgG₃ antibodies of the subject to an HIV antigen, wherein elevated IgG₃ immunoreactivity indicates that the subject has recently been infected with HIV.

As the skilled addressee would appreciate, and as supported by the Examples provided herein, the precise timeframe in which IgG₃ immunoreactivity to HIV antigens is elevated will vary somewhat between subjects. This, at least in part, can be attributed to standard variations between individuals, the quantity of virus in the initial infection, and/or the subject's immune or treatment status at the time of infection. However, the present inventors have shown that IgG₃ immunoreactivity is elevated at least at some point of time during the period from about 34 days to about 120 days post-infection. Thus, as used herein with regard to the first aspect, a “recent” HIV infection refers to at least some point of time during the period from about 34 days to about 120 days post-infection.

As used herein, the term “about 34 days” refers to a range of at least 29 to 39 days. Furthermore, as used herein, the term “about 120 days” refers to a range of at least 104 to 136 days.

By “immunoreactivity of IgG₃ antibodies of the subject to an HIV antigen”, or similar term, is meant the level of IgG₃ in, for example, a sample obtained from a bodily fluid such as serum, plasma, whole blood, urine or saliva that is capable of specifically binding to an antigen of HIV.

The findings of the present inventors are also useful for incidence testing a population to characterize the occurrence and stage of HIV infections within the population.

Thus, in a second aspect, the present invention provides a method of determining the incidence of HIV infected individuals in a population, the method comprising determining the immunoreactivity of IgG₃ antibodies from individuals of the population to an HIV antigen, wherein the proportion of individuals having elevated IgG₃ immunoreactivity is indicative of the incidence of HIV in the population.

Preferably, as outlined above with regard to the first aspect, the method further comprises performing a second assay to detect HIV, such as but not limited to, an assay that detects HIV infection at a stage of infection other than the incident IgG₃ immunoreactivity assay. By using at least two assays which detect HIV infections at different stages post infection more accurate population data are obtained.

The word “population” shall be taken to mean a group of people according to their race, country of origin, socio-economic condition, sex, sexual orientation, age, religion, employment, health, etc. Preferably, the population is a population for which there are insufficient, accurate data on the incidence of HIV infection.

As will be apparent to those skilled in the art of epidemiology, it is not necessary to assay every member of a population to obtain the incidence of a particular character. Accordingly, a sufficient number of individuals of the population to provide a statistically significant estimate of the population is tested, and as a consequence, the actual numbers to be tested is readily determined without undue experimentation. Preferably, the individuals tested are randomly selected from the population.

Previous researches have developed an assay, generally known as the “p24 antigen assay”, that detects an HIV infection from about 15 days post infection. However, this assay generally does not detect HIV infections up to about 120 days post-infection. Thus, to detect an HIV infection between about 15 days to about 120 days post infection the p24 antigen assay is combined with that of the present invention.

Accordingly, in a third aspect, the present invention provides a method of distinguishing between a recent and an established HIV infection in a subject, the method comprising determining the immunoreactivity of IgG₃ antibodies of the subject to an HIV antigen, and determining the levels of p24 antigen in the subject, wherein elevated IgG₃ immunoreactivity and p24 antigen levels indicate that the subject has recently been infected with HIV.

Preferably, p24 antigen levels are determined by an enzyme immunoassay (EIA).

Preferably, the method of the third aspect detects HIV infection in a window of about 15 to about 120 days post-infection.

The present inventors have found that elevated IgG₃ immunoreactivity to HIV antigens can also be detected in an established infection, albeit at a reduced level when compared with the initial peak following infection. Thus, this immune response could be considered as a general marker for HIV infection. However, when conducting such epidemiological studies, particularly when the HIV infection status of the subject is unknown, it may be necessary to perform an independent analysis that detects established HIV infections. This second assay would guard against low levels of IgG₃ in an established HIV infection being identified as a false negative.

Thus, in a preferred embodiment, the method further comprises determining if the subject has an established HIV infection. Methods for identifying established HIV infections are well known in the art. Any of such methods may be used inconjunction with the methods of the present invention and include, but are not limited to, detection using nucleic acid testing techniques e.g. the polymerase chain reaction or variations thereof, and analysing other immunoglobulin immunoreactivity, such as total IgG binding to various HIV antigens.

Preferably, the method is performed on a sample obtained from the subject, such as but not limited to, serum, whole blood, urine, PBMC, saliva or Ig fraction. Preferably, the sample was obtained previously from the subject, such as, for example, by a consulting physician who has referred the sample to a pathology laboratory for analysis.

As outlined above, it has been found that the IgG₃ immune response to an HIV infection is a general marker for such an infection.

Thus, in a fourth aspect the present invention provides a method for detecting an HIV infection, the method comprising determining the immunoreactivity of IgG₃ antibodies of the subject to an HIV antigen, wherein IgG₃ immunoreactivity indicates that the subject is infected with HIV.

In the context of the present invention, “antigen” includes a full length HIV protein, a derivative of a full-length HIV protein, such as but not limited to, a protein fragment or a synthetic peptide that comprises an amino acid sequence corresponding to a part or parts of a full-length HIV protein, including any modified fragment or synthetic peptide having a ligand attached thereto. The term “antigen” also includes an analogue of a full-length HIV-protein or an analogue of a fragment or peptide, such as but not limited to, a peptide mimetic having a different amino acid sequence to the HIV protein or fragment or peptide, or a non-peptide molecule, that binds to the same IgG₃ antibodies as the HIV protein.

Preferably, the HIV antigen comprises a protein selected from the group consisting of p24, p66, p32, p17, gp160, an IgG₃ antigenic fragment thereof, or combination fragments thereof. More preferably, the HIV antigen comprises p24 (also referred to herein as the “capsid protein”) or an IgG₃ antigenic fragment thereof.

IgG₃ immunoreactivity can be measured using any technique known in the art. Such techniques include, but are not limited to, enzyme immunoassays (EIA) including enzyme-linked immunosorbent assays (ELISA), Western and other immunoblots, radioimmunoassays (RIA), radioimmunopreciptation assays (RIPA), particle agglutination assays and immunofluorescence assays (IFA). Preferably, IgG₃ immunoreactivity is measured using an EIA. Preferably, the EIA is an ELISA.

As known in the art, the level of IgG₃ immunoreactivity will be measured with varying sensitivity depending on the detection system used. Thus, the level of IgG₃ immunoreactivity required to be considered as “elevated” in accordance with the present invention will depend upon the precise procedure utilized. A particular procedure for measuring IgG₃ immunoreactivity to an HIV antigen can be tested against samples obtained from individuals known to be recently infected with HIV (preferably between about day 34 to about day 120 post infection) and compared with similar samples obtained from individuals who have an established HIV infection such as, but not limited to, individuals who are known to have been infection for at least 1 year or so. Upon comparison of the results, a suitable level of IgG₃ immunoreactivity can be determined which readily distinguishes a recent infection as defined herein (which has “elevated IgG₃ immunoreactivity”) from an established infection. As an example, an EIA system utilizing a mouse monoclonal anti-human IgG₃ linked to horseradish peroxidase to detect IgG₃ antibodies which bind p24 can be used to identify elevated IgG₃ immunoreactivity to p24, wherein elevated levels of immunoreactivity are defined by at least 0.5 absorbance units at an optical density of 405 nm in this specific assay format. Assay variation can be controlled by using the value from a standard curve which corresponds to 0.5 absorbance units this being approximately 20 μg/ml of anti-p24 IgG₃. Considering the present disclosure, the skilled addressee could readily use standard techniques to determine a suitable “cut-off” to be used to define “elevated IgG₃ immunoreactivity” when using other methods of detecting IgG₃ immunoreactivity to an HIV antigen.

Preferably the method further comprises transferring the results of the method onto a record medium. Preferably, the record medium is selected from the group consisting of paper and an electronic medium such as a computer disk. In this embodiment, the “results” may be the level of IgG3 immunoreactivity detected and/or whether the subject has no HIV infection or a recent or established HIV infection.

In one embodiment, a sample can be preadsorbed with protein A (which binds all human IgG isotypes except IgG₃). Any reagent capable of binding human IgG could then be used for detection purposes (not necessarily an IgG₃ specific antibody). By preadsorbing first the assay could also be performed using any reagent that could bind antibody (eg. protein G) as a capture and then detecting the bound IgG₃ with labelled antigen (eg. HRP-p24).

In a fifth aspect, the present invention provides for the use of an agent which specifically binds IgG₃ for the detection of an HIV infection in a subject.

Preferably, the agent is an anti-IgG₃ antibody.

Preferably, the agent is used to detect a recent HIV infection.

The HIV can be any strain or isolate. Preferably, the HIV is selected from the group consisting of HIV-1 and HIV-2.

Preferably, the subject is a human.

In a sixth aspect, the present invention provides a kit for detecting an IgG₃ antibody in a sample which specifically binds an HIV antigen, the kit comprising an HIV antigen, and an agent which specifically binds the IgG₃ antibody. When in use the agent specifically binds the IgG₃ antibody which in turn specifically binds the HIV antigen.

Preferably, the agent is a protein. More preferably, the agent is an anti-IgG₃ antibody, or IgG₃ binding fragment thereof.

Anti-IgG₃ antibodies, and methods for the production thereof, are known in the art. Preferably, the anti-IgG₃ antibody is an anti-human anti-IgG₃ antibody. Such anti-human anti-IgG₃ antibodies include, but are not limited to, goat anti-human anti-IgG₃ antibody, rabbit anti-human anti-IgG₃ antibody and mouse anti-human anti-IgG₃ antibody.

Preferably, the agent is detectably labelled. More preferably, the detectable label is an enzyme, a fluorescent molecule, a radio-active isotope or a chemiluminescent molecule.

Preferably, the kit further comprises a purified human IgG₃ antibody. In this instance, the IgG₃ antibody can be used to construct a standard curve which aids in quantifying the levels of IgG₃ immunoreactivity in a sample tested using the kit of the invention.

Preferably, the HIV antigen comprises a protein selected from the group consisting of p24, p66, p32, p17, gp160, an IgG₃ antigenic fragment thereof, or combination fragments thereof. More preferably, the HIV antigen comprises p24 or an IgG₃-antigenic fragment thereof.

By “specifically binds” is meant that the agent recognizes and physically interacts with IgG₃ and does not significantly recognize and interact with other antigens in the sample being tested.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention will hereinafter be described by way of the following non-limiting Figures and Examples.

### BRIEF DESCRIPTION OF THE ACCOMPANYING FIGURES

FIG. 1. Detection of anti-HIV-1 antibodies by total IgG and IgG isotype specific Western blot. The blots have been probed with a polyclonal anti-human IgG secondary antibody, a monoclonal anti-human IgG₁ antibody or a monoclonal anti-human IgG₃ antibody. The strips within each panel were incubated with sequential plasma samples taken from a recently infected individual. The estimated number of days post HIV infection that the sample was obtained is indicated below each strip and the HIV-1 specific protein bands are identified to the left of the strips. The day of infection was estimated as 15 days before the date of presentation with acute retroviral syndrome.

FIG. 2. Detection of anti-p24 IgG₃ antibodies by Western blot in 17 HIV-1 seroconversion panels. The net intensity of the p24 band for each sample within the 17 panels are overlayed in the plot to demonstrate the presence of a peak of IgG₃ reactivity to p24 which occurs early after infection, mainly before 120 days. The net intensity of each band is plotted against the estimated day post-infection that the sample was taken (symbols). Serial bleeds from a single patient are connected by solid lines. The day post-infection was estimated by designating the day of presentation with acute retroviral syndrome or the first sample to produce a p24 antigen reactive result as day 15.

FIG. 3. Detection of anti-p24 IgG₃ antibodies by EIA in 17 HIV-1 seroconversion panels. The results are overlayed in the above plot to demonstrate the presence of a strong transient IgG₃ response to p24 extending approximately between days 34 to 120. post-infection. The absorbance values obtained at 405 nm are plotted against the day post-infection (symbols) that the blood was taken and serial bleed from a single patient are connected by solid lines. The day post-infection was estimated by designating the day of presentation with acute retroviral syndrome or the first bleed to produce a p24 antigen reactive result as day 15.

FIG. 4. IgG₃ anti-p24 and p24 antigen reactivity in 17 HIV-1 seroconversion panels are shown. Each panel of samples collected from a single individual is represented by a line across time (days). The circles represent the days on which a sample was taken from each individual. The black circles represent samples which produced a positive IgG₃ response in the anti-p24 EIA. A positive result was defined as an absorbance of greater than 0.5 at 405 nm. The grey circles represent samples which produced a positive p24 antigen result as interpreted according to the manufacturers' instructions.

### DETAILED DESCRIPTION OF THE INVENTION

General Techniques

Unless otherwise indicated, the recombinant DNA and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present), and are incorporated herein by reference.

General Methods for Detecting an HIV Infection

Many techniques have been developed for detecting an HIV infection. At least some of these procedures are commercially available in “kit” form. Many of the techniques are generally described in HIV: A Practical Approach (Volume 1: Virology and Immunology. Ed. Jonathan Karn, IRL Press); AIDS Testing: A comprehensive guide to technical, medical, social, legal, and management issues (Ed. Gerald Schochetman and J. Richard George. 2^{nd} Edition. Springer-Verlag, 1994); Gallo et al. (1986) and Mylonakis et al. (2000). An overview of at least some of these techniques is provided below. Furthermore, at least some of these techniques, including those of the claimed invention, can readily be adapted to be performed using nanocrystals such as those described in WO 00/27365, U.S. Pat. No. 6,207,392, Nolan and Sklar (2002), and Han et al. (2001).

Enzyme Immunoassay (EIA) or Enzyme-Linked Immunosorbent Assay (ELISA) Methodology

ELISA detection systems have been used routinely in EIAs for many years in the detection of HIV infection by showing the presence of anti-HIV antibodies. Furthermore, there are many licensed manufacturers of EIAs and ELISAs for detecting antibody to HIV. The sensitivity of third generation EIAs is close to 100 percent when any anti-HIV-antibody is present in peripheral blood. However, these assays cannot differentiate between the earliest stages of infection and established infection.

Basically, EIA methodology involves the following steps. HIV-antigens are purified from viral lysate, prepared by recombinant DNA technology or peptide synthesis and are coated onto the wells of microwell plates or onto other matrixes such as beads to form the “solid phase” of the assay. The serum of an individual is added to the well. Antibody, if present, reacts with the antigen, and the other well contents are then washed away. An indicator reagent consisting of an anti-human antibody bound to an enzyme or other detection system is added to the well. If the serum contained HIV-specific antibodies, these will remain attached to the solid-phase antigen, and the enzyme-conjugated anti-human antibody will attach to these antibodies and thus to the solid phase. Another washing step follows. If the individual's serum contains antibody to HIV, the enzyme remains attached through antibody to the solid phase and is available to catalyze a colour-producing reaction when an appropriate substrate is added to the well. The colour change is measured in a spectrophotometer. Absorbance values above a cut-off value calculated from control samples are considered reactive.

This basic methodology has been adapted to encompass a wide variety of assay formats including, both antigen and antibody capture assays as well as antigen and antibody competition assays.

Immuno Transfers—Western Blots

Western blots are another form of EIA which have been commonly used for establishing the presence of true anti-HIV antibodies. Several commercially produced kits are available. The limitations of the procedure are largely due to the variety of antigens and subjective interpretations used by different laboratories.

Basically, the technique involves the following steps. Crude or semi purified HIV viral lysate often spiked with purified recombinant proteins is layered onto an SDS polyacrylamide gel and then the proteins are separated by electrophoresis. The viral proteins (the HIV-antigens) migrate through the molecular pores of the gel at rates determined by electrical charge, molecular weight and protein folding; the higher molecular weight proteins migrate more slowly and form bands closer to the starting point. The proteins in the gel are then transferred (“blotted”) to a nitrocellulose membrane. The membrane may be cut into thin strips, each consisting of all of the separated viral protein antigen bands. A single test strip is incubated with a dilution of a test sample or a control and then washed and incubated with a labelled (tagged) anti-human immunoglobulin. At this point, the procedure is similar to any other indirect immunoassay. The label is usually an enzyme (for example, horseradish peroxidase or alkaline phosphatase) that will react with a specific substrate to produce an insoluble coloured precipitate which appears as a band on the strip wherever there is an antigen-antibody complex. Alternatively chemiluminescent substrates provide far greater sensitivity. Reaction with a positive serum sample produces a pattern of bands on the strip that is characteristic of HIV. Detailed investigations have confirmed the identity of all the viral specific bands.

This technique has been widely modified resulting in a vast number of assay formats that may be used in a large variety of situations. Recombinant proteins or peptides can be “painted” directly onto nitrocellulose membranes resulting in highly reproducible strips containing equivalent amounts of the relevant antigens. Strips or dot blots produced in this way provide the basis for many of the rapid diagnostic tests including cartridge and dip stick tests.

Particle Agglutination Assays

Antigen or antibody labeled latex particles, sepharose, polyurethane microcapsules, colloidal gold or red blood cells have been employed to produce a wide range of immuno-agglutination assays. Particles can be obtained commercially with a large range of surface chemistries allowing for great flexibility when coupling them to either antibody or antigen. These techniques are typically used in rapid assay formats that are usually scored visually, but are also quite easily adapted to automation.

Immunofluorescence Assay (IFA)

The IFA for HIV-antibody is more technically demanding and more expensive than Western blots. Because virtually all the antigens present in an infected cell are available for reaction with the test specimen, it is a very sensitive assay. It is a procedure familiar to many laboratories because it is used for detecting antibodies to a wide variety of viral and bacterial antigens.

Basically, the technique involves the following steps. A suspension of a lymphocyte cell culture infected with HIV is placed on a microscope slide, air-dried, and fixed in acetone or methanol. Uninfected control cells are added to the suspension or put in separate spots on the slide to provide a means for detecting non-specific reactions (fixed slides can be made in large batches and stored frozen or desiccated.) Diluted test sera are added to the cell spots, the slide is washed, incubated again with fluorescein-conjugated anti-human globulin, washed again, and then inspected for fluorescein fluorescence using an ultraviolet microscope.

Typical localized fluorescence of infected cells occurs after reaction with positive sera. Little or no fluorescence occurs with negative sera. Non-specific reactions (such as those caused by antinuclear antibody) are recognized because of fluorescence in uninfected control cells.

Radioimmunoprecipitation

The radioimmunoprecipitation assay is used primarily in research. It is generally too technically demanding for routine use in clinical laboratories. Radioimmunoprecipitation is especially sensitive for antibodies to the higher molecular weight major envelope glycoproteins gp160 and gp120, which some Western blot techniques miss. The principle of RIPA involves competitive binding of radiolabeled antigen and unlabeled antigen to a high-affinity antibody. The antigen is generally labelled with a gamma-emitting isotope such as ¹²⁵I. The labelled antigen is mixed with antibody at a concentration that just saturates the antigen-binding sites of the antibody molecule, and then increasing amounts of unlabeled antigen of unknown concentration are added. The antibody does not distinguish labelled from unlabeled antigen, and so the two kinds of antigen compete for available binding sites on the antibody. With increasing concentrations of unlabeled antigen, more labelled antigen will be displaced from the binding sites. By measuring the amount of labelled antigen free in solution, it is possible to determine the concentration of unlabeled antigen.

Nucleic Acid Testing (NAT)

The sensitivity and versatility of the NAT technology have been exploited extensively in developing approaches for the diagnosis of infectious diseases. A number of approaches which use NAT to quantify the amount of HIV nucleic acid in clinical samples have been described, examples include those described in Rogers et al. (1989), Mulder et al. (1994) and Piatek et al. (1993). In general DNA testing is used as a screening assay to identify infected samples. These DNA assays are often qualitative and more sensitive than the quantitative RNA tests which are used to monitor a patient's response to infection or treatment.

Polymerase Chain Reaction (PCR)

PCR can detect HIV nucleic acid sequences in a DNA form, as proviruses integrated into the chromosomes of infected cells or as RNA synthesized in infected cells that are actively expressing virus or within virus particles present in the cell-free plasma. PCR can amplify HIV DNA either from detergent lysates of infected cells or from purified cells.

One example is Reverse Transcription Polymerase Chain Reaction (RT-PCR). At least one RT-PCR HIV assay system is commercially available through Roche Ltd. To accurately quantify the cDNA present at the start of PCR (reflecting the number of copies of HIV RNA in the original plasma specimen), the RT-PCR method uses a synthetic competitive RNA template (an identical DNA competitive template is used when the assay is applied to measure the amount of HIV RNA in a clinical specimen) that is derived from HIV sequences but differs from the real viral RNA by virtue of an internal deletion. Titered, known quantities of this competitive HIV RNA template are added to a series of PCR assay mixtures, each holding identical amounts of the test sample containing an unknown quantity of the HIV target sequence. The competitive template is included in the reverse transcription and amplification steps. Following PCR the amount of the respective products is quantified by EIA.

Branched DNA Assay

The branched DNA assay (bDNA) strategy to measure the amount of HIV RNA in plasma of infected patients uses highly sensitive branched DNA probes to detect and quantify viral RNA sequences (Cao et al., 1995; Urdea et al. 1993). At least one bDNA HIV assay system is commercially available through Chiron Corporation.

In this non-PCR-based method, sensitivity derives from signal amplification rather than the target amplification that provides the basis for PCR. To measure HIV RNA copy number in plasma by the bDNA assay, the sample is first centrifuged to pellet the virus particles. The pelleted virus is then lysed with a solution containing detergent and proteases. This crude extract containing HIV RNA is added to microtiter wells coated with oligonucleotide probes homologous to conserved sequences in the HIV genome. The viral RNA forms duplexes with the probe sequences and is thus captured. After the well is washed, bDNA amplifier molecules are hybridized to the bound HIV RNA, and then alkaline phosphatase probes that bind to the bDNA amplifier molecules are added. The HIV-specific bDNA probes are thus labeled with an enzyme that catalyzes the release of a chemiluminescent moiety from its substrate (dioxetane), and the amount of the signal (light) produced is quantified using a luminometer.

Measuring IgG₃ Immunoreactivity

IgG₃ immunoreactivity can be measured using any technique known in the art including, but are not limited to, EIAs including ELISAs, immunoblots including Western blots, radioimmunoassay (RIA), radioimmunopreciptation assay (RIPA), particle agglutination assays and immunofluorescence assay (IFA). Essentially, each of the above-mentioned immunological detection systems can be specifically adapted for determining the presence or levels of anti-HIV IgG₃ immunoreactivity in a sample.

In a particularly preferred embodiment, label-conjugated anti-human antibody (generally referred to in the art as the “secondary antibody”) which is specific for IgG₃, and hence does not bind other immunoglobulin isotypes, is used to detect the immunoreactivity of IgG₃ antibodies to an HIV antigen.

Such a secondary antibody includes intact molecules as well as fragments thereof, such as Fab, F(ab′)2, and Fv which are capable of binding the epitopic determinant. These antibody fragments retain some ability to selectively bind with its antigen and are defined as follows:

(1) Fab, the fragment which contains a monovalent antigen-binding fragment of an antibody molecule can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab′)2, the fragment of the antibody that can be obtained by treating whole antibody with the enzyme pepsin without subsequent reduction; F(ab)2 is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody (“SCA”), defined as a genetically engineered molecule containing the variable region of the light chain, the variable region of the heavy chain, linked by a suitable polypeptide linker as a genetically fused single chain molecule.

Methods of making these fragments are known in the art (see for example, Harlow and Lane, Antibodies: A Laboratory Manual, Cold Spring Harbor Laboratory, New York (1988), incorporated herein by reference).

Examples of immunoassays—particularly Western blots and ELISA's—for detecting IgG₃ immunoreactivity are disclosed herein.

p24 and the Detection Thereof

Following infection with HIV there is an antigenemia as the virus multiplies in the lymphoid systems and circulates unchecked prior to seroconversion. Circulating viral antigens (including HIV p24) are present in the serum of all infected persons. Once an individual's immune system generates specific antibody to HIV p24, HIV p24 antigen usually becomes undetectable because the majority is bound to antibody and viral suppression results in decreased production of antigens. p24 antigen assays are known in the art, examples include those described by von Sydow et al (1988), Busch et al. (1995), Ly et al. (2001), and U.S. Pat. No. 5,641,624. HIV p24 antigen and anti-HIV p24 antibody detection assays are available as ELISA kits from a number of commercial sources. The detection of HIV p24 antigen and anti-HIV p24 antibody by these kits are carried out separately, either in separate kits (e.g., Abbott) or in separate wells (e.g., Dupont HIV-1 p24 Core Profile ELISA, and Coulter HIV-1 p24 Antigen Assay System).

The amounts of HIV p24 core protein (p24 antigen) present in serum, plasma, or tissue culture medium can be measured using an antigen capture enzyme-linked immunosorbent assay (ELISA). In this assay, samples containing HIV are treated with a detergent to disrupt virus particles, and added to microtiter wells or mixed with polystyrene beads that are coated with specific anti-p24 “capture” antibodies. Following a washing procedure, the bound HIV p24 protein is detected by specifically chosen polyclonal or monoclonal anti-p24 antibodies, which are in turn detected by anti-immunoglobulin antibodies or may be directly coupled to an enzyme. The anti-immunoglobulin antibodies used are conjugated with an enzyme that cleaves a specific substrate, releasing a coloured product that can be measured spectrophotometrically. By measuring the intensity of the colour produced, one can determine quantitatively the amount of HIV p24 antigen present.

Kits

Kits of the present invention may comprise a packaged combination of reagents in predetermined amounts with instructions for performing a method of the invention. When the anti-IgG₃ antibody is labeled, the kit may comprise suitable reagents for detecting the label. For example, when the label is an enzyme, the kit will include substrates and cofactors required by the enzyme (e.g., a substrate precursor which provides the detectable chromophore or fluorophore). In addition, other additives may be included such as stabilizers, buffers and the like. The relative amounts of the various reagents may be varied widely to provide for concentrations in solution of the reagents which substantially optimize the sensitivity of the assay. The reagents may be provided as dry powders, usually lyophilized, including excipients which on dissolution will provide a reagent solution having the appropriate concentration.

HIV antigen for use in kits of the present invention can be provided/obtained from any source known in the art. For example, HIV antigen can be produced using recombinant methods such as those described herein. Alternatively, HIV antigen can be purchased from a commercial supplier, for instance p24 can be obtained from Biodesign International (Maine, USA).

### EXAMPLE

Materials and Methods

BCIP/NBT phosphatase substrate system was purchased from Kirkegaard & Perry laboratories Guildford UK. HIV-1 viral lysate was obtained from Zeptometrix Corporation NY, USA. All electrophoresis and Western transfer apparatus and reagents as well as the DC and Bio-Rad protein assays were supplied by Bio-Rad Laboratories, Hercules, Calif., USA. ExtrAvidin-alkaline phosphatase, purified human IgG₃, biotinylated mouse monoclonal anti-human IgG₁ and anti-IgG₃ were from Sigma Chemical Co. Ltd., St. Louis, Mo., USA. Biotinylated goat anti-human IgG was purchased from Biosource International. Nickel agarose resin was from Qiagen, Victoria, Australia. SeeBlue Plus 2 pre-stained standards were obtained from Invitrogen, Australia. HRP conjugated mouse monoclonal anti-human IgG₃ was from Southern Biotechnology Assoc. Inc. Birmingham, Ala. All other reagents were purchased from BDH, Dorset, England.

Serum Samples and Panels

Seven anti-HIV-1 seroconversion panels were purchased—three from Boston Biomedica, Inc., MA, USA (Panel 4-6: A, AG and W), three from Impath-BioClinical Partners Inc., Ma, USA (Panels 1-3: BCP#62357, BCP#65522 and BCP#60772) and one from Bio-Rad (Panel 7: RP-018). Panels 8 to 17 were obtained from the Center for Immunology, St. Vincent's Hospital, NSW. One hundred negative control samples were obtained from Australian Red Cross Blood Service, Victoria and ten long-term, positive control samples were from The Carlton Clinic Pty. Ltd., Victoria. One hundred and twenty three samples from 55 individuals identified with false positive p24 bands on Western blot (all with follow-up bleeds taken three months later that confirmed their status as anti-HIV-1 negative) were obtained through the National Serology Reference Laboratory, Melbourne, Australia's testing program.

Isotype Specific Western Blot

HIV-1 viral lysate was sonicated three times for 10 seconds, aliquoted and stored frozen at −70° C. An optimum concentration of 250 μg of total protein per gel was established by running a range of HIV-1 lysate concentrations varying between 150 and 350 μg/gel. The gels were probed using a standardised dilution series generated from an HIV-1 positive plasma sample and the intensity of the bands was compared with a gel prepared using a previously optimised batch of HIV-1 viral lysate. The 250 μg of HIV-1 viral lysate was loaded on a 12% SDS-PAGE and run at 200V until the tracking dye had travelled 13 cm. The proteins were transferred to nitrocellulose (0.45 μm pore size) over-night at 30V.

Western blot strips were blocked on a shaker for 2 hours at room temperature in 0.3% skim milk powder in TBS (Tris buffered saline pH 7.4). Samples were diluted 1 in 100 in 0.1% skim rnilk powder in TBS and incubated at room temperature for 4.5 hours. Strips were washed three times in TBS/T (Tris buffered saline containing 0.05% Tween 20) and incubated for 1 hour at room temperature in a 1 in 1000 dilution of the appropriate biotinylated secondary isotyping antibody diluted in 0.1% skim milk powder. Strips were again washed three times in TBS/T before the addition of a 1 in 5000 dilution of ExtrAvidin-alkaline phosphatase in 0.1% skim milk powder in TBS for 30 minutes at room temperature. Strips were washed extensively in TBS/T and the BCIP/NBT phosphatase substrate system was used to visualise bands. The reaction was stopped by extensive washing in water and air-dried over-night at room temperature.

Bands were analysed using a Kodak DC290 zoom digital camera and Kodak 1D image analysis software. Quantification of the band densities was performed using “net intensity” which is pre-defined in the program as the sum of the background subtracted pixel values in the band rectangle.

Production of p24 Protein

The p24 encoding region of HIV-1 was cloned into the bacterial expression vector pET16 behind a 5′ region encoding a 10 amino acid histidine tag. The purified construct was transfected into BL21DE3 cells and grown in Luria-Bertani Medium (LB medium) containing 1% glucose and 100 μg/ml Ampicillin. On obtaining a culture density of 0.7 at OD₆₀₀ expression was induced with 1 mM IPTG (isopropylthio-β-D-galactoside) for 4 hours at 37° C. following which, the cells were pelleted and frozen at −70° C.

Each pellet (derived from 1 liter of culture) was resuspended in 40 ml of binding buffer (20 mM Tris-HCl pH8.0 containing 0.5M NaCl and 5 mM imidazole) and passed twice through a cell crusher. The homogenate was spun at 20,000 g for 20 min at 4° C. and the supernate (diluted 1:2 with binding buffer) was applied to 15 ml of nickel agarose equilibrated in binding buffer. The column was washed extensively with 20 mM tris-HCl pH8.0 containing 1M NaCl and 25 mM imidazole, then eluted with 20 mM tris-HCl pH8.0 containing 500 mM NaCl and 1M imidazole. The protein containing fractions were pooled using the Bio-Rad protein assay and the final protein concentration determined using the Bio-Rad DC protein microassay against a γ-globulin standard curve ranging from 0.2 to 1.5 mg/ml.

Bacterial expression and protein purification of p24 were analysed on 15% SDS-PAGE mini-gels (Laemmli et al. 1970) stained with Coomassie blue G250 and a duplicate gel was transferred to nitrocellulose and probed with a 1 in 200 dilution of human plasma known to contain antibodies to HIV-1 p24. The Western transfer was probed for anti-human IgG as described above.

Anti-p24 Specific ELISA

A standard curve for IgG₃ was constructed over the range 1000 to 0.5 ng/well in 96 well ELISA plates by using serial dilutions of purified human IgG₃ in 0.2M sodium carbonate buffer pH9.6. This dilution series was coated overnight at 37° C. onto the wells of each 96 well ELISA plate in duplicate in a total volume of 50 μl. The aspirate was then tested for the presence of residual unbound IgG₃ in an anti-human IgG₃ specific ELISA and by using the Bio-Rad DC protein micro assay. Both assays confirmed 100% binding of IgG₃ to the ELISA plate wells over the entire linear region of the standard curve between an absorbance range of 0.0 to 2.5 at OD₄₀₅ₙₘ (ie. all concentrations≦2.22 μg/ml).

The optimum p24 coating concentration was determined by incubating a range of antigen concentrations in a final volume of 50 μl of 0.2M sodium carbonate buffer pH 9.6 in 96 well plates overnight at 37° C. Antigen was used over a range from 5.0 to 0.005 μg of p24/well to establish an optimum coating concentration of 0.5 μg/well. At this concentration the wells were saturated with antigen and as a result produced a maximal absorbance curve for samples titrated out to dilutions between 1 in 10 and 1 in 10⁹.

Following overnight coating with 0.5 μg of antigen per well the ELISA plates were washed twice in PBS/T (phosphate buffered saline pH7.4 containing 0.1% Tween 20). Each well was blocked with 150 μl of blotto (50 mM Tris-HCl pH8.0 containing 5% skim milk powder, 2 mM calcium chloride, 80 mM sodium chloride and 0.2% nonidet P40) for 2 hours at 37° C. Plates were washed twice with PBS/T and plasma samples were diluted 1 in 10 in blotto. Five fold serial dilutions of samples were made in blotto and 100 μl of each dilution was added per well and incubated at 37° C. for 1 hour. Plates were washed three times in PBS/T. A mouse monoclonal anti-human IgG₃ horseradish peroxidase conjugate was diluted 1 in 1000 in blotto and 100 μl added per well. The plates were incubated at 37° C. for 1 hour and then washed four times in PBS/T. ABTS (2 mM 2,2′-azino-bis(3-ethylbenzthiazoline-6-sulfonic acid) in 25 mM sodium citrate buffer pH4.5) containing 0.3% hydrogen peroxide was used as a substrate for the HRP conjugated secondary antibody by adding 100 μl per well and incubating at room temperature for 20 minutes. The reaction was stopped with 50 μl of 5% oxalic acid and the absorbance of the wells read at an OD of 405 nm.

Results

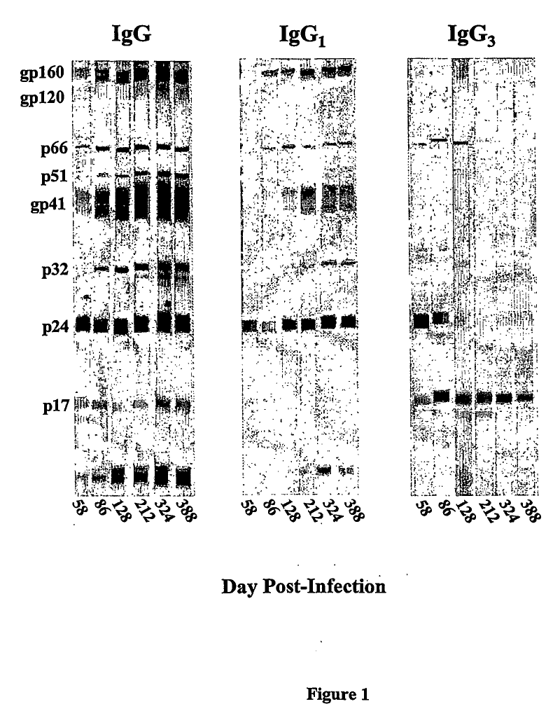

In order to characterise the humoral immune response to HIV-1 infection Western blots using isotype specific secondary antibodies were performed. FIG. 1 illustrates a typical Western blot profile obtained using serial bleeds from an HIV-1 infected individual. Each strip corresponds to a plasma sample taken at an estimated 58 days, 86 days, 128 days, 212 days, 324 days and 388 days post HIV-1 infection. The date of infection was estimated as 15 days prior to the date of presentation with acute retroviral syndrome. In FIG. 1, the strips were probed with a biotinylated polyclonal anti-human IgG secondary antibody, a biotinylated monoclonal anti-human IgG₁ antibody or a biotinylated monoclonal anti-human IgG₃ was used as a secondary antibody.

The major response observed to HIV-1 was an IgG response (FIG. 1) and the major antibody isotype contributing to this broad response was IgG₁. IgG₁ was produced in response to all the HIV-1 bands routinely scored on Western blots. These encompass the majority of proteins transcribed from the gag, pol and env genes. IgG₃ was directed primarily towards the gag derived proteins p17 and p24 with variable and somewhat transient responses to the pol derived proteins p66 and p32 (FIG. 1). In addition to positive and negative controls the isotype specific Western blot was also run against 123 samples that had previously produced false positive p24 bands. Repeat bleeds had been obtained from these individuals to confirm their status as anti-HIV-1 negative. None of these samples showed any IgG₃ immunoreactivity to p24 (results not shown).

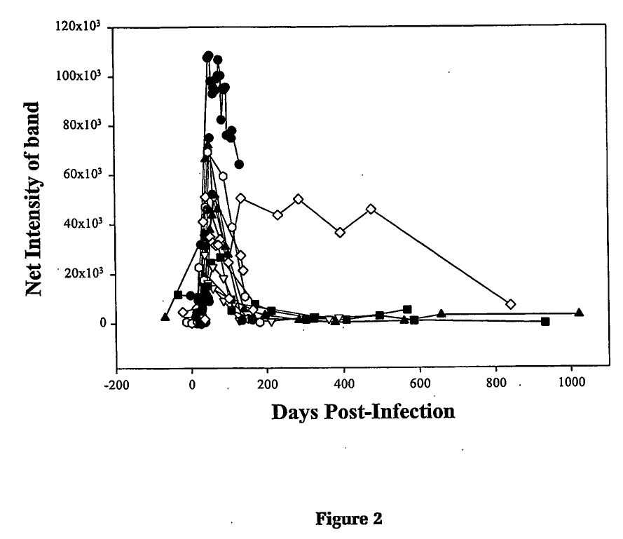

Qualitative Western blot results give a comprehensive picture of the maturation of the humoral immune response to HIV-1 antigens. By using densitometry these results can be semi-quantified to produce an impartial representation of the trends observed. Western blot strips were analysed using the digital image obtained from a Kodak camera and the net intensity of the bands was calculated using Kodak 1D analysis software. By plotting the net intensity of the bands against the day post infection the major observation was an early transient IgG₃ response to the gag derived protein p24 (FIG. 2). The solid lines in FIG. 2 represent consecutive bleeds from each individual donor while the symbols represent the estimated number of days post-infection that the sample was obtained. This IgG₃ response to p24 did not completely disappear with time but a definite increase in the net intensity of bands was observed between days about 34 to about 120 post-infection. More specifically, the combined results indicated a window of detection of 34±5 days to 120±16 days post-infection.

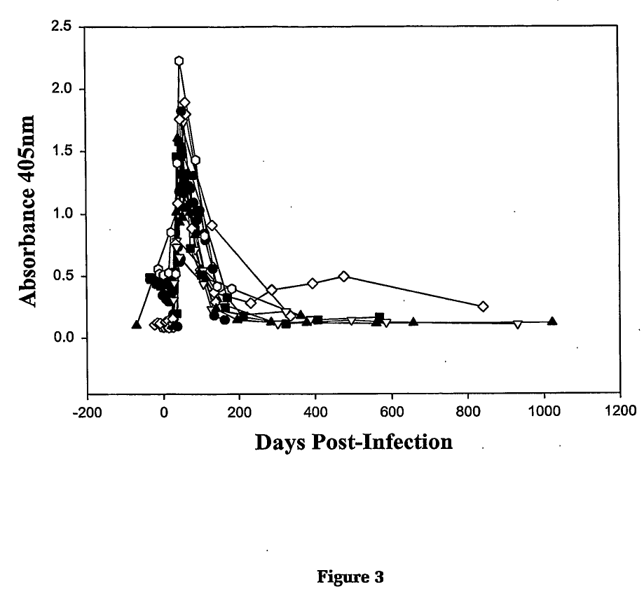

Western blots provided a good overall picture of what was occurring during the maturation of the immune response to HIV-1, but because of their inferior sensitivity, other techniques that could provide a sensitive quantitative result were necessary to confirm this qualitative assay. For this reason p24 was cloned, expressed, purified and used as the antigen to coat plates for an IgG₃ specific ELISA. In FIG. 3 the lines represent serial bleeds from individuals while the symbols indicate the day post-infection that the sample was taken. The “day post-infection” was estimated by designating the day of presentation with acute retroviral syndrome or the first bleed to produce a p24 antigen reactive result as day 15. The y-axis represents the absorbance at 405 nm obtained in the anti-p24 IgG₃ specific ELISA and the x-axis shows the day post-infection. FIG. 3 illustrates the strong peak of IgG₃ activity directed towards p24 between days 34 to 120 post infection in all of the seroconversion panels analysed.

To determine the specificity of this response the inventors applied this assay to 100 negative control samples obtained from healthy blood donors, 10 long-term HIV-1 positive individuals and 123 samples obtained from 55 individuals who had produced a consistently false positive p24 band by Western blot. The average absorbance value obtained from the 100 negative control samples was 0.11 with a standard deviation of 0.04 and a range of 0.078 to 0.425 absorbance units at 405 nm. The average absorbance obtained from 10 positive samples from long term infected individuals was 0.14 with a standard deviation of 0.04 and a range of 0.106 to 0.221. The mean absorbance obtained on the 123 samples that had produced a false p24 band by Western blot was 0.095 with a standard deviation of 0.035 and a range of 0.068 to 0.263.

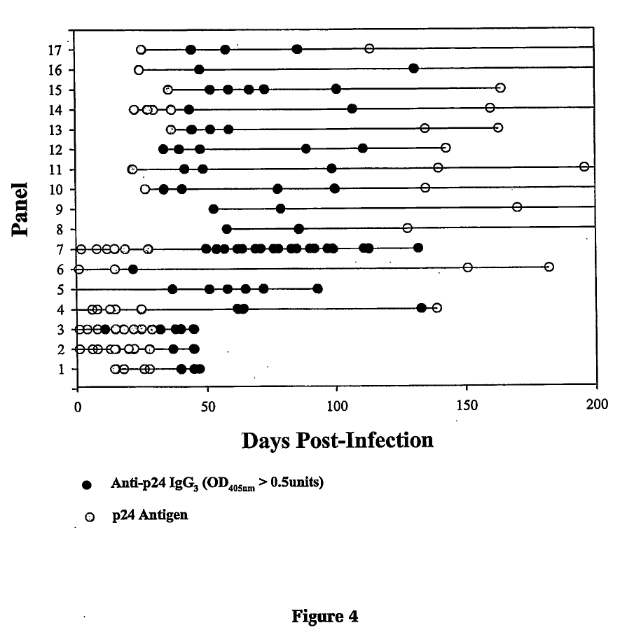

The data obtained from the IgG₃ specific p24 ELISA indicated that given an appropriate cut-off it may be possible to identify samples falling within this 34 to 120 day window period where the peak of IgG₃ activity was occurring. An arbitrary cut-off of 0.5 absorbance units at an optical density of 405 nm was assigned and classified samples as positive if they fell above this value. In FIG. 4 the solid lines represent the 17 HIV-1 panels and the circles indicate the day post-infection that the sample was obtained during the 0 to 200 day window period of interest. All samples taken prior to day 0 were negative and all samples post day 200 produced absorbances of less than the cut-off. Samples that produced a positive IgG₃ response to p24 (i.e. >0.5 absorbance units) are shown as solid black symbols in FIG. 4 while samples that produced an absorbance of <0.5 A₄₀₅ are shown as open symbols.

It was accepted that the primary response to HIV-1 infection would not be an IgG₃ response and as a result an assay that could detect HIV-1 positive samples prior to day 34 was required. A p24 antigen assay was chosen to identity samples that were taken from individuals infected with HIV-1 but not yet displaying an IgG₃ anti-p24 response. Samples that were p24 antigen positive are shown as pink circles in FIG. 4. Panel 1 was the only panel where a non-reactive gap was observed between the presence of p24 antigen and the detection of anti-p24 IgG₃ antibodies. There was also a gap between the presence of p24 antigen and the detection of antibody reported by Bioclinical Partners in the specification sheets provided with this panel. During this period when protein based assays produced negative results, RNA levels dropped from 25 000 copies/ml to undetectable levels by the Chiron bDNA assay. Despite no detectable p24 antigen during this period the drop in RNA corresponded with an increase in antibody levels. The remaining 14 panels all displayed an overlap between the presence of p24 antigen and the appearance of anti-p24 IgG₃ antibodies.

Discussion

The present inventors have found that a transient elevation in the IgG₃ response to p24 occurs early after infection and is maintained throughout a period of 34 to 120 days post-infection in untreated individuals.

To control for any assay variation due to batch to batch differences in reagents the standard curve can be used as an internal control. When absorbance values are converted to relative antibody concentration by reading the values directly from the standard curve, plate to plate, day to day and batch to batch variations are eliminated.

In order to detect HIV infected individuals prior to about 34 days post infection an additional assay can be performed in parallel. Current antibody assays become positive approximately 22 days after infection (Busch et al. 1995). The use of a p24 antigen assay would extend that window period out to approximately 15 days post-infection. A testing algorithm, which includes the use of an HIV RNA PCR, would provide a window period of approximately 11 days after an individual becomes infected.

To demonstrate that a dual testing strategy would be useful in detecting HIV infected patients without missing individuals who have not fully seroconverted. The inventors combined a p24 antigen assay with an anti-p24 IgG₃ specific ELISA and found that sequentially collected samples from between day 15 to 120 post HIV infection could be identified. This combination of assays provides a means of reliably identifying recently infected HIV individuals and distinguishing them from individuals with an established infection.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed, particularly in Australia, before the priority date of each claim of this application.

### REFERENCES

Binley, J. M. et al. (1997) AIDS Res. and Hum. Ret. 13:1007-1015.
Busch, M. P. et al. (1995) Transfusion 35:91-97.
Cao, Y. et al. (1995) AIDS Res. Hum. Retrov. 11:353-361.
Chargelegue, D. et al. (1993) Clin. Exp. Immunol. 93:331-336.
Chargelegue, D. et al. (1995) J. Infect. Diseases. 172:897-898.
Gallo, D. et al. (1986) J. Clin. Microbiol. 23:1049-1051.
Gouws, E. et al. (2002) J. Acquir. Immune. Defic. Syndr. 29:531-535.
Han, M. et al. (2001) Nat. Biotech. 19:631-635.
Janssen, R. S. et al. (1998) JAMA. 280:42-48.
Laemmli, U.K. (1970). Nature. 277:680-685.
Ly, T. G. et al. (2001) J. Clin. Microbiol. 39:3122-3128.
Mulder, J. et al. (1994) J. Clin. Microbiol. 32:292-300.
Mylonakis, E. et al. (2000) Am. J. Med. 109:568-576.
Nolan, J. P. and Sklar, L. A. (2002) Trends Biotech. 20:9-12.
Piatek, M. Jr et al. (1993) Biotechniques 14:70-81.
Rogers M. F. et al. (1989) N. Eng. J. Med. 320:1649-1654.
Urdea, M. S. et al. (1993) AIDS 7:S11-14.
von Sydow, M. et al. (1988) Br. Med. J. 296: 238-240.

1. A method of distinguishing between a recent and an established HIV infection in a subject, the method comprising determining the immunoreactivity of IgG3 antibodies of the subject to an HIV antigen, wherein elevated IgG3 immunoreactivity indicates that the subject has recently been infected with HIV.

2. The method of claim 1, wherein the IgG3 immunoreactivity is elevated at least at some point of time during the period from about 34 days to about 120 days post-infection.

3. A method of determining the incidence of HIV infected individuals in a population, the method comprising determining the immunoreactivity of IgG3 antibodies from individuals of the population to an HIV antigen, wherein the proportion of individuals having elevated IgG3 immunoreactivity is indicative of the incidence of HIV in the population.

4. A method of distinguishing between a recent and an established HIV infection in a subject, the method comprising determining the immunoreactivity of IgG3 antibodies of the subject to an HIV antigen, and determining the levels of p24 antigen in the subject, wherein elevated IgG3 immunoreactivity and p24 antigen levels indicate that the subject has recently been infected with HIV.

5. The method of claim 4, wherein the IgG3 immunoreactivity is elevated at least at some point of time during the period from about 15 days to about 120 days post-infection.

6. The method of claim 4, wherein the p24 antigen levels are determined by an enzyme immunoassay.

7. The method according to claim 1 which further comprises determining if the subject has an established HIV infection.

8. The method of claim 7, wherein an established HIV infection is determined by detecting HIV derived nucleic acids or proteins in the subject.

9. A method for detecting an HIV infection, the method comprising determining the immunoreactivity of IgG3 antibodies of the subject to an HIV antigen, wherein IgG3 immunoreactivity indicates that the subject is infected with HIV.

10. The method according to claim 1, wherein the HIV antigen is selected from the group consisting of: p24, p66, p32, p17, gp160, an IgG3 antigenic fragment thereof, or combination fragments thereof.

11. The method according to claim 1, wherein IgG3 immunoreactivity is determined using a technique selected from the group consisting of: enzyme immunoassays (EIA), Western blots, radioimmunoassays (RIA), radioimmunopreciptation assays (RIPA), particle agglutination assays and immunofluorescence assays (IFA).

12. The method of claim 11, wherein IgG3 immunoreactivity is determined using an EIA.

13. The method of claim 12, wherein the EIA is an enzyme-linked immunosorbent assay (ELISA).

14. The method of claim 12, wherein elevated IgG3 immunoreactivity is at least 0.5 absorbance units at an optical density of 405 nm or a value obtained from a standard curve which corresponds to this absorbance or approximately 20 μg/ml p24 specific IgG3.

15. The method according to claim 1, wherein the method is performed on a sample obtained from the subject.

16. Use of an agent which specifically binds IgG3 for the detection of an HIV infection in a subject.

17. The use of claim 16, wherein the agent is an anti-IgG3 antibody.

18. A kit for detecting an IgG3 antibody in a sample which specifically binds an HIV antigen, the kit comprising an HIV antigen, and an agent which specifically binds IgG3 antibody.

19. The kit of claim 18, wherein agent is an anti-IgG3 antibody.

20. The kit of claim 18, wherein the agent is detectably labelled.

21. The kit according to claim 18, wherein the kit further comprises an IgG3 antibody which specifically binds to the HIV antigen.
